(19) United States
(12) Patent Application Publication
(10) Pub. No.: US 2025/0288740 A1
(43) Pub. Date: Sep. 18, 2025

(54) SYSTEM AND METHODS TO CUSTOMIZE MODES INTO ACTIONABLE PUMP SETTINGS

(71) Applicant: INSULET CORPORATION, Acton, MA (US)

(72) Inventors: Joon Bok LEE, Acton, MA (US); Yibin ZHENG, Hartland, WI (US); Matthew ALLES, Mooresville, NC (US); Jason O’CONNOR, Acton, MA (US)

(21) Appl. No.: 19/035,369

(22) Filed: Jan. 23, 2025

Related U.S. Application Data
(60) Provisional application No. 63/566,445, filed on Mar. 18, 2024.

(51) Int. Cl. A61M 5/172; G16H 20/17

(52) U.S. Cl. CPC A61M 5/1723; G16H 20/17; A61M 2205/505; A61M 2230/201

(57) ABSTRACT

A system and device for establishing a customized mode of a medicament delivery algorithm. The device may include a processor configured to receive an indication to establish a custom mode. The device implements an initial learning phase. A processor executing the initial learning phase receives and evaluates sensor data for a duration of time. A sensor operable to make analyte measurements may provide analyte measurement values. Based on a result of the initial learning phase, the device sets medicament delivery algorithm parameters for the custom mode, thereby establishing the custom mode for operation of a wearable automated medicament delivery system.

### CROSS-REFERENCE TO RELATED APPLICATIONS

This application claims priority to and the benefit of U.S. Provisional Application No. 63/566,445, filed Mar. 18, 2024, the entirety of which is incorporated herein by reference.

### BACKGROUND

Automated drug delivery systems that utilize drug delivery algorithms typically allow users to execute manual adjustments to their medicament delivery settings to account for temporary changes in life patterns that may lead to different medicament sensitivities. The manual adjustments may include modifications to the target glucose concentrations, basal medicament delivery, and other clinical parameters, or other adjustments, and the breadth of the possible adjustments may intimidate some users.

Some automated drug delivery systems may offer one or two temporary modes to facilitate or case this manual adjustment by making standardized setting change recommendations; however, the range of changes to each user's medicament delivery, particularly insulin delivery requirements, carry significant interpersonal variations which mean singular, fixed settings of such life pattern adjustments may not be optimal for these users.

In addition, there are insulin delivery systems that have default modes of operation, such as an exercise mode or a sleep mode. These default modes have default algorithm setting values that are pre-set or have setting values within a narrow pre-set range may be suboptimal for a large percentage of users.

### BRIEF SUMMARY

In one aspect, a device for establishing a customized mode of a medicament delivery algorithm is disclosed and the device may be configured to receive an indication to establish a custom mode. The device may implement an initial learning phase. The processor executing the initial learning phase receives and evaluates sensor data for a duration of time. Based on a result of the initial learning phase, the device sets medicament delivery algorithm parameters for the custom mode, thereby establishing settings for the custom mode that may be re-initiated in the future without having to go through another initial learning phase.

In another aspect, a system for establishing a custom mode is provided. The system may include at least one sensor, a wearable medicament delivery device, and a processor. The at least one sensor may be configured to generate information related to a user. The wearable medicament delivery device may be configured to deliver medicament to the user. The processor may be configured to receive an indication to establish the custom mode, evaluate information provided by the at least one sensor to establish parameter settings of the custom mode, and generate medicament delivery algorithm parameters for the custom mode. The parameters settings may be used by a medicament delivery algorithm that controls the delivery of the medicament to the user when in the custom mode.

### BRIEF DESCRIPTION OF THE DRAWINGS

To easily identify the discussion of any particular element or act, the most significant digit or digits in a reference number refer to the figure number in which that element is first introduced.

FIG. 1A illustrates an exemplary process for customizing a medicament delivery algorithm in accordance with the disclosed subject matter.

FIG. 1B illustrates an exemplary set of graphical user interfaces for setting up a custom mode set up process in accordance with the disclosed subject matter.

FIG. 2 illustrates an exemplary custom mode set up process in accordance with the disclosed subject matter.

FIG. 3A illustrates an exemplary step in a process of selecting a preset activity in accordance with the disclosed subject matter.

FIG. 3B illustrates an exemplary step in a process of selecting a preset activity following the step in FIG. 3A in accordance with the disclosed subject matter.

FIG. 3C illustrates another exemplary step in a process of selecting a preset activity in accordance with the disclosed subject matter.

FIG. 3D illustrates an exemplary presentation of a selected preset activity in accordance with the disclosed subject matter.

FIG. 4 illustrates an exemplary medicament delivery system operable to implement the examples disclosed herein.

### DETAILED DESCRIPTION

Better processes and techniques for creating and/or setting a customized mode of use for individual users are needed instead of the “one setting fits all” modes provided by some automated drug delivery systems.

Described herein are systems and methods to allow users of MDA systems to establish customized “modes,” regardless of the type of activity, and for the algorithm of the medicament delivery algorithm (MDA) systems to dynamically adjust future interactions of the same mode by estimating changes to the user's insulin needs following each modal interaction. Operating modes, such as sleep and exercise modes, are typically pre-defined with “rule of thumb” clinical or a manufacturer's settings.

In the known systems, the system may allow users to select a fixed variation in the aggressiveness of the algorithm's insulin delivery to compensate for expected changes in the user's insulin needs. For instance, the system may allow the user to select a fixed “Activity mode” over a set duration that may incorporate a higher target glucose concentration and/or reduced maximum delivery limits, for example, to decrease mean insulin delivery during the period.

However, appropriate adjustment of the system's settings may vary significantly depending on the user and their activities. For instance, an “activity” may be a high-intensity exercise for one user versus a short walk for another user—each requiring different optimal adjustments to the algorithm's settings. However, in the algorithm described herein, the personal definition of “activity” may be customized and determined after a threshold duration of user participation in the “activity” and after evaluating recorded sensor data from the user's participation in the “activity.”

The disclosed system can allow a user to select one mode for use during some period of time during the day, and, for the rest of the day, the algorithm can operate under its normal algorithmic settings according to user specific settings, such as target glucose setpoint, operational settings (e.g., maximum insulin delivery, minimum insulin delivery, insulin sensitivity, and the like), and so on.

In addition, the novel custom mode set up process utilizes minimal inputs, most frequently one input, from the user to initiate the custom mode set up process. In this manner, customization to the user is possible without requiring the user to enter a plethora of adjustments, which may intimidate some users.

In one example of the custom mode set up process, the name of the activity is the only input obtained from the user, and the user does not have to input any medicament delivery algorithm parameter settings. For example, when a user is about to start an activity in which the user desires to be governed by a custom mode, the user may first set up a custom mode by inputting a name for an activity mode (e.g., biking, swimming, or the like) representing something that is identifiable by the user and which may be selected in the future by the user, but is not based on pre-established manufacturer settings. As the user participates in the activity, a processor in a device of the medicament delivery system may log data related to the user's physiological state and/or metabolic state (e.g., glucose measurement values, ketone measurements, heart rate, or the like) during the particular activity, for example, by tagging such data as corresponding to the particular activity, compare such activity data to the user's physiological and/or metabolic state before and/or after the particular instance or duration of this particular activity, and may further compare data logged for this particular activity instance to data logged during prior instances of the named activity. The duration of the activity may be measured either by a period of time (e.g., minutes or hours) or by a set number of data points (e.g., number of sensor (e.g., glucose) measurement values, such as 12, 24, 36, or 48, and/or other physiological or metabolic measurement values) as described with reference to later examples.

When using medicament delivery systems today, even automated delivery systems implementing an algorithm, the user is commonly forced into a set of fixed parameters (e.g., a default blood glucose setpoint, default glucose rate of change, a default minimum or default maximum insulin delivery, or the like) when the user selects one of a predetermined default modes offered by the system, such as an “activity” mode as mentioned above. The fixed parameters are merely predetermined parameter settings that are different from the “normal” operating mode. For example, in response to an indication that the user is selecting a default “activity” mode, an algorithm may select a higher default glucose setting, such as 140 mg/dL, or the like.

It may be helpful at this time to differentiate a custom mode from the “normal” operating mode of an MDA that controls delivery of medicament to a user. A “normal” operating mode may be a daily mode that is implemented as the user goes about their daily life, or engages in routine, daily activities that most people engage in, or a mode that is implemented during non-strenuous activity, which makes up the vast majority of time of an average user (e.g., wakes up, cats breakfast, goes to work, school, or the like, cats lunch, finishes day at work, school, or the like, goes home, plays with the dog, visits with spouse, kids, friends, etc., cats dinner, reads a book, watches television or surfs the Internet, winds down, and goes to bed). The medicament delivery algorithm parameter settings for the normal mode may have set values, for example, a target glucose level, a target ketone level, or the like. The MDA is configured to adjust medicament delivery to operate within the values set for the medicament delivery algorithm parameters. For example, when the user is eating a snack or a meal, or engaging in other daily activities, the parameter settings may be pre-configured to address those daily activities so as to keep the user's analyte levels within a target range for at least a majority of the time. However, if the user decides to engage in an atypical activity, the user's medicament delivery algorithm parameter settings may not be appropriate to maintain the user's blood glucose within the target analyte range.

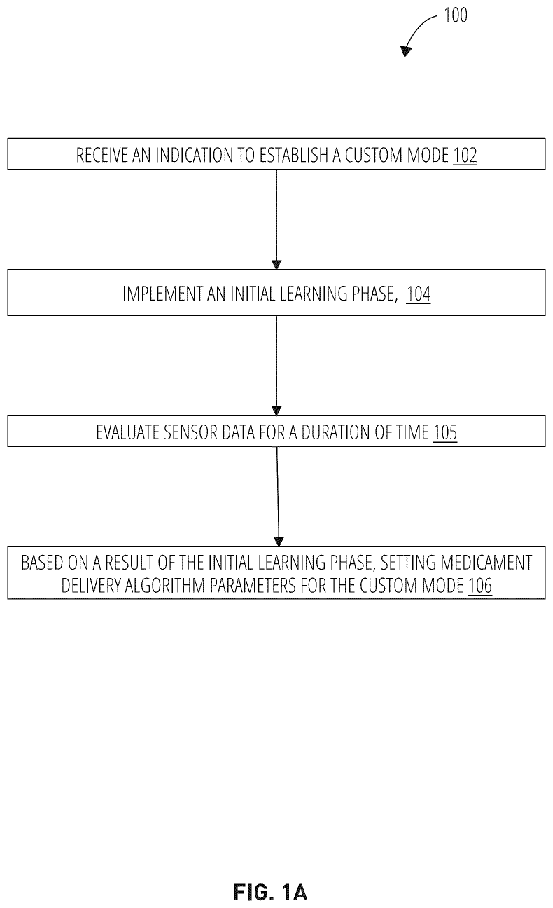

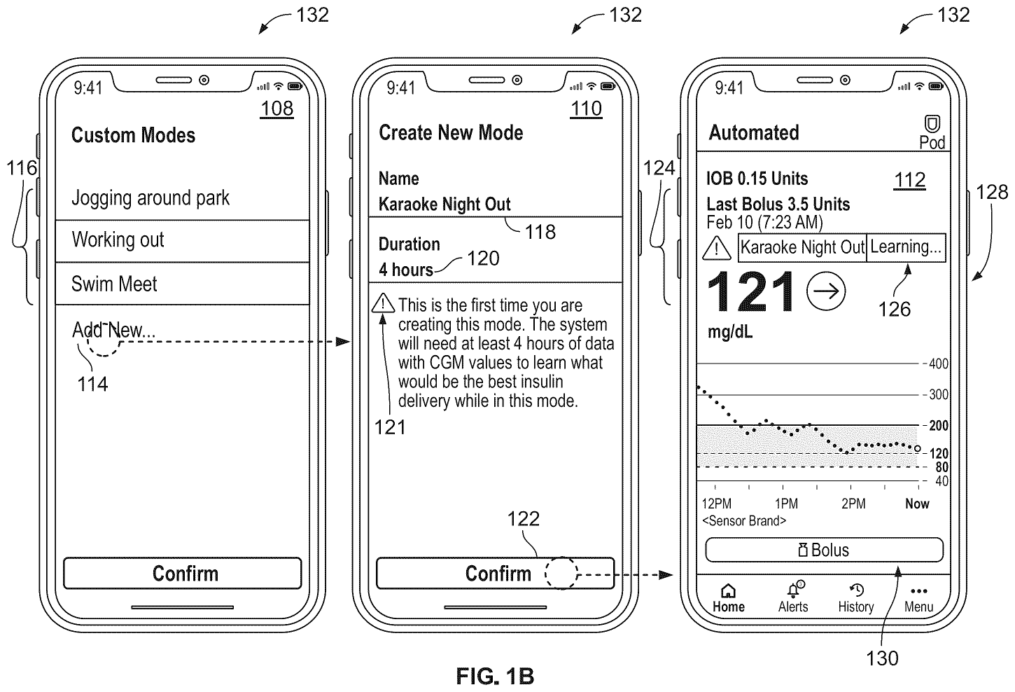

In contrast to a “normal” operating mode, a custom mode may be used for activities that the user does not regularly engage in (e.g., on a daily basis), or that may impact a user's analyte levels more than the user's normal or typical activities. A custom mode set up process may initially utilize the medicament delivery algorithm parameter settings that have been established for the “normal” mode, and evaluate sensor data for a set duration, and based on the result of the evaluation, derive the performance of the MDA. It may be helpful to describe an exemplary custom mode set up process with respect to the flowchart of FIG. 1A.

FIG. 1A illustrates an exemplary process for customizing a medicament delivery algorithm in accordance with the disclosed subject matter.

During typical operations of the medicament delivery algorithm, sensor data is recorded nearly continuously and may be stored onboard a device (e.g., a controller, a wearable medicament delivery device, smart accessory device, or the like) for a period of time, such as 4 hours, 6 hours, 8 hours, 24 hours, 48 hours, or the like. Alternatively, the recorded sensor data may be distributed between devices, such as the wearable medicament delivery device and a controller, may be operable to store hours or days of sensor data, and upload it to a controller, which may store several hours, days or weeks of sensor data, and then upload to a cloud-based service, or the like.

As part of the custom mode set up process, the system utilizes a period of learning during which sensor values are recorded as recorded sensor data to enable the processor to evaluate the recorded sensor data and set values for medicament delivery algorithm parameter settings. It may be helpful to explain the concept of the time related to the learning phase, which is referred to as the learning phase duration, and the time period outside of the learning phase. The “time outside the learning phase” may not be considered a learning phase time period. For example, if a user initiates a custom mode set up process at 4 μm and the learning phase duration is 4 hours, the learning phase lasts from 4 pm until 8 μm. The time from 4 μm to 8 pm is considered the learning phase, while the time before 4 μm and after 8 pm is considered time that is the outside the learning phase time period. The “time outside the learning phase” time period can extend for several hours (e.g., 12, 24, 48, 72, or the like) surrounding or on either side of (i.e., before or after) the learning phase.

Examples of time that may comprise the outside the learning phase time period may include several hours before the learning phase and several hours after the learning phase. The several hours before and after do not have to be equal. Or the system may begin using the time when the learning phase duration ends as the time for the outside the learning phase time period. The processor may be operable to record data from sensors (i.e., recorded sensor data, for example) during the learning phase and during the outside the learning phase time period.

The process 100 may be implemented in programming code (i.e., software), hardware firmware, or a combination thereof. The programming code may be stored in a memory and is executable by a processor.

In step 102, a processor executing process 100 receives an indication to establish a custom mode. For example, the indication may be received either prior to the user giving or after the user gives a customized mode name to the custom mode to be established. The indication may be for example a user input, e.g., the user pressing a button on a graphical user interface.

In step 104, process 100 implements an initial learning phase, wherein a processor executing the initial learning phase receives and evaluates sensor data during the learning phase for a duration of time, e.g., while the user is engaging in the activity associated with the particular custom mode. The initial learning phase may be a first learning phase for the particular custom mode. For some particular custom mode's, the initial learning phase is the only learning phase. For other particular custom modes, the initial learning phase is a first learning phase with subsequent learning phases occurring each time the particular custom mode starts or is entered by the user.

In an example, the duration of time is a default duration of time set by the system. In another example, the duration of time is received from the user via an input device. The duration of time may be the same as the learning phase duration, and the terms may be used interchangeably herein. In an operational example, when receiving an indication of a duration of the initial learning phase, the processor may receive either the default duration of time (e.g., 2, 4, 5 or 6 hours) from memory for the initial learning phase, or may receive the duration of time via the input device from the user (e.g., the user inputs an amount of time). In examples, in which the processor receives a duration of the initial learning phase from the user, the processor may be operable to replace or reset the default duration of time for the initial learning phase with a duration of the initial learning phase indicated by the MDA. In an example, the processor may maintain the default duration of time for the initial learning phase regardless of the duration received via the user input because the default duration is an amount of time needed to collect a sufficient amount of recorded sensor data. A sufficient amount of recorded sensor data may be considered a recorded data threshold.

During the learning phase, the MDA operates as it normally would be based on the medicament delivery algorithm parameter settings for the time of day at which the learning phase occurs. For example, the medicament delivery algorithm parameter settings are not changed when the custom mode is being established. The MDA is operable to collect data from one or more sources, such as a continuous glucose monitor, a fitness device, a ketone sensor, a heart rate monitor, blood oxygen sensor, a pedometer, analyte sensor, or the like. The collected and recorded sensor data may be categorized based upon the sensor that provided the data, and the sufficiency of the respective recorded sensor data may be dependent upon the sensor that provided the data. For example, the sensor data may be of different categories of data, such as a quantity of data points received from a continuous glucose monitor, the number of hours over which the continuous glucose monitor received data, a quantity of data points received from a ketone sensor, glucose values or ketone values and/or trends from the continuous glucose monitor, time stamps of when data is received, ketone values and/or trends from the ketone sensor, time stamps of when data is received, a number of steps provided by the pedometer, sensor values, such as a heart rate level and trend, oxygen levels, and the like, and data from each respective sensor may be categorized and processed (e.g, normalized, etc.) differently.

In an alternative learning phase, the MDA either in response to receipt of the indication to establish the custom mode at step 102 or implementation of the initial learning phase at step 104, the processor may be configured to enable a user to select medicament delivery algorithm initial settings for the custom mode. For example, prior to implementing the initial learning phase at step 104, the user may be prompted to set these medicament delivery algorithm initial settings. Alternatively, if no MDA settings are received in response to the prompt, the processor at step 104 may automatically select default MDA settings. These default settings may be the same parameter settings that the MDA would use when in a normal operating mode.

The evaluation performed at step 105 of the received sensor data may include a process, such as comparing with respect to reference data related to the custom mode. For example, the processor may compare data gathered during the learning phase to data gathered outside the learning phase, and determine what parameter settings may need to change based on a result of the comparison.

In another operational example, during the learning phase, the processor may be operable to record the sensor data provided by one or more sensors related to an activity covered by the custom mode. The processor executing the MDA is continuously recording data, which is stored. However, when a custom mode is being set up as in the process 100, the recorded sensor data may also be flagged as being associated with the custom mode being set up, or, if memory capacity is sufficient, the recorded sensor data may be duplicated. The processor may be operable to determine whether the amount of recorded sensor data is sufficient to recommend a setting value for one or more parameters or all parameters in the list of medicament delivery algorithm parameters. The period over which the learning phase occurs is long enough to allow a sufficient amount of sensor data to be recorded to allow the MDA to make appropriate medicament delivery algorithm parameter settings. The sufficiency of the recorded sensor data may be determined by the number of data points or values recorded during the learning phase, or by an amount of time that has elapsed since the start of the learning phase. An example of a number of data points or values recorded may be 72 measurements in the learning phase, which may equate to 6 hours of learning phase time, and such measurements may include glucose measurements, heart rate, blood oxygen readings, or the like. Other examples of learning phase durations may include 2, 4, 5, 8 hours, or the like, but preferably at least 4 hours as reflected in the infographic 121 in FIG. 1B.

The programming code may configure the processor to evaluate the recorded sensor data to establish parameter settings for the activity of the custom mode. Based on the evaluation, the established custom mode may provide or be populated with a list of medicament delivery algorithm parameters that are optimized to the user's medicament metabolism. Recommended settings may be included for each of the medicament delivery algorithm parameters in the list of medicament delivery algorithm parameters and may be based on the result of the evaluation.

In step 106, the processor, based on a result of the initial learning phase, is operable to set the medicament delivery algorithm parameters for the custom mode. In addition, the processor executing the process 100 programming code may be operable to store the set medicament delivery algorithm parameters in association with the given mode name.

As a result, the established custom mode has medicament delivery algorithm parameter settings that are optimized for the user when participating in an activity that is associated with the established or named custom mode, and the system may deliver medicament (or not deliver medicament) based on the updated algorithm parameter settings for the custom mode.

In addition, the medicament delivery algorithm parameter settings determined for the established custom mode may be updated whenever the established custom mode is operable. For example, upon starting the established custom mode, whatever the duration of operation, the processor may initiate a subsequent learning phase based on the recommended medicament delivery algorithm parameter settings.

FIG. 1B illustrates an exemplary set of graphical user interfaces for a custom mode set up process in accordance with the disclosed subject matter.

A user device 132 may be operable to present a number of graphical user interfaces, such as a first graphical user interface 108, a second graphical user interface 110, and a third graphical user interface 112, that enable a user to implement a custom mode set up process, such as part or all of the process described in the example of FIG. 1A as well as those described in later examples.

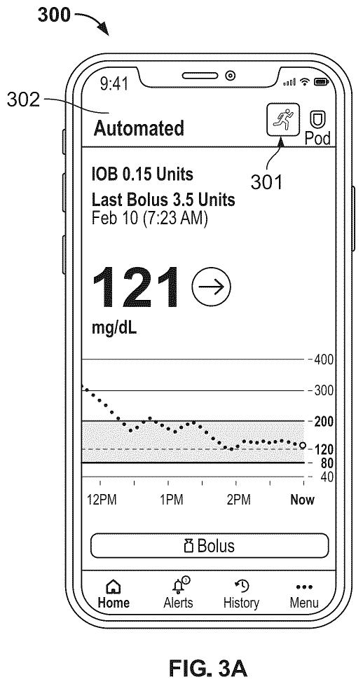

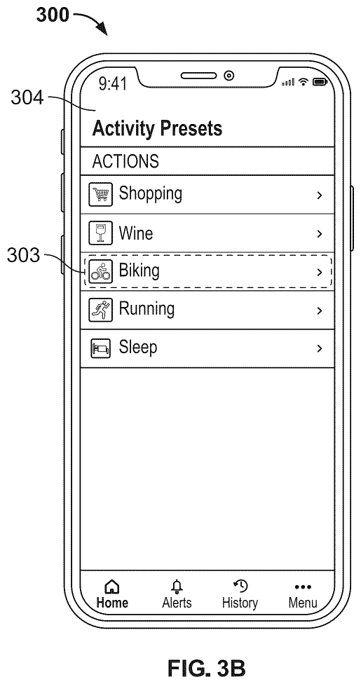

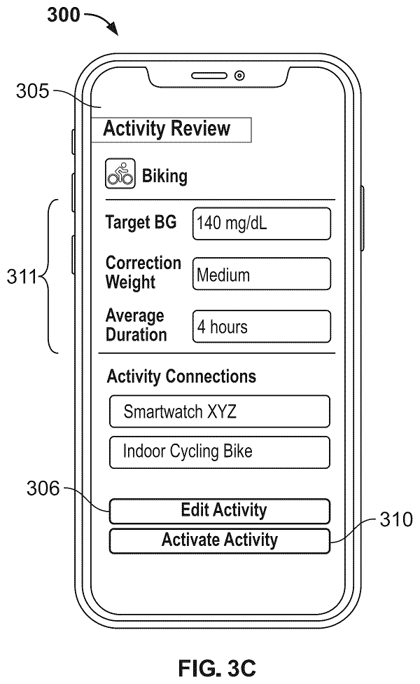

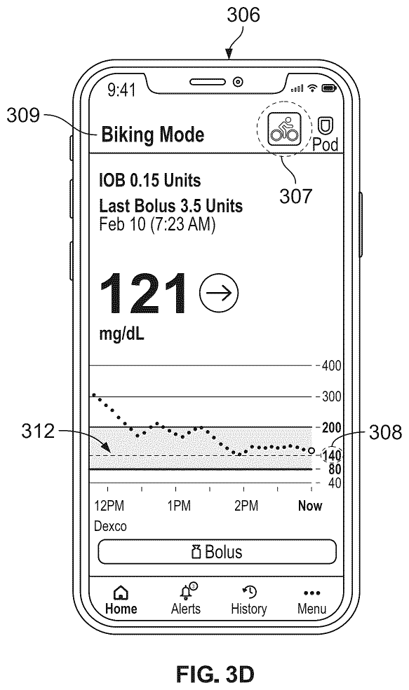

In an example, upon being presented with a custom mode's menu, such as that shown in first graphical user interface 108, the user has an option to select other custom modes 116 previously set up, or may set up a new custom mode by, for example, selecting an Add New button 114. The other custom modes 116, such as jogging around park, working out, or swim meet, may be custom modes that may have already been configured utilizing a custom mode set up process as described herein, and that remain stored in a memory of the user device 132. In the example, the user may wish to set up a new custom mode by selecting the Add New button 114. After selection of the Add New button 114, the first graphical user interface 108 may transition to presentation of the second graphical user interface 110. In the second graphical user interface 110, the custom mode may be named by the user inputting via an input device, such as a microphone or a keyboard, the custom mode name 118 (i.e., Karaoke Night Out). In the second graphical user interface 110, the default or average duration 120 that the user would typically desire to engage in the custom activity may be set. In this case, the user sets a “karaoke night out” duration of 4 hours. This custom activity duration may or may not correspond to a learning phase duration. As explained above, the learning phase duration is a period of time or a time corresponding to an amount of data that enables the processor to record enough recorded sensor data for evaluation. For example, this learning phase duration may preferably be at least 4 hours, as reflected on graphical user interface 110 at 121. If the user's desired custom activity duration is less than the learning phase duration, then the user may be required to engage in the custom activity more than once to allow sufficient time to gather sufficient sensor data for evaluation. In this example, the user's desired duration of 4 hours corresponds to a preferred minimum amount of learning phase duration, so the custom activity mode settings may be established after the user participates in the custom activity one time (e.g., in four hours), assuming the user goes the full desired duration of 4 hours and does not suspend the activity early. Once the default or average activity duration 120 is set, a custom mode set up confirmation 122 may be highlighted or presented for selection by a user. It should be noted that this average or default duration 120 may be adjusted in the future when the user engages in the custom activity again. For example, as reflected in FIG. 3C, a duration of 4 hours was previously set for the “Biking” activity. The user may adjust this time based on the duration of time the user intends to actually engage in the activity. It should be noted that the user may also suspend or stop the activity after starting or activating the activity—for example, if the user ends their biking, or karaoke, or other custom activity earlier than the default time period or the specified time period entered.

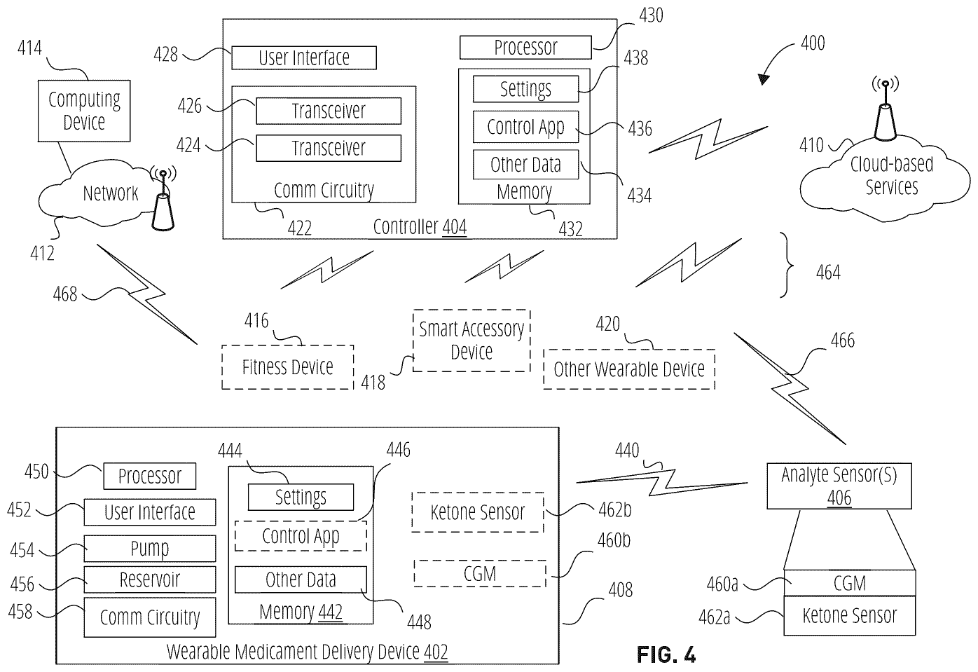

As the user engages in the new custom activity and the learning phase progresses, a third graphical user interface 112 may be presented on the user device 132. The third graphical user interface 112 may present recorded data 124. The recorded data 124 may include an amount of insulin on-board (IOB), a number of Units delivered in a last bolus (including time and/or date of the bolus), a current analyte sensor measurement value, which may be a most recent glucose measurement and/or ketone measurement, a learning phase status indicator 126, and an analyte chart 128. The learning phase status indicator 126 may indicate how much time elapsed since initiation of the new custom activity. Alternatively, the learning phase status indicator 126 may indicate how much data has been collected with respect to a recorded data threshold. The recorded data threshold may be, for example, a number of glucose measurements that have been collected. The number of glucose values may not reflect the time since the learning phase has started (e.g., 12 analyte values received may not correspond to 60 minutes of time) because some values from the analyte sensor may not have been properly received by the user device 132, or the controller 404 or medication delivery device 402, for example, as shown in FIG. 4. In such cases, the data threshold may take longer to achieve. The processor may also be configured to present a learning phase status indicator 126 that informs a user that analysis or learning is ongoing, for example, using a bar graph as shown, or presenting a numerical status such as a percentage of learning or duration completed or remaining, a display of a degree learned, or an amount of analysis (in minutes or hours) that may still need to be performed.

In addition, the analyte chart 128 may also be presented to show the user how the activity for which the custom mode is being established affects the user's analyte being monitored, such glucose measurement values, ketone measurement values, or the like, while the medicament delivery algorithm is using non-custom mode parameter settings. The analyte chart 128 may also present a high boundary glucose measurement value as the upper line (solid horizontal), a target glucose measurement value as the center line (larger dashed line), a low boundary glucose measurement value as the lower line (smaller dashed line), a timeline, and a number of glucose measurement values collected during the learning phase.

A medicament delivery request button or bolus button 130 may also be presented on the third graphical user interface 112 that enables a user to request delivery of medicament.

The user device 132 may also include a processor, memory, and communication circuitry such as that described in more detail with reference to other examples that facilitate the processes described with reference to FIGS. 1A and 1B.

As previously mentioned, the established custom mode has medicament delivery algorithm parameter settings that are optimized for the user when participating in the activity associated with the established or named custom mode. For example, after the learning phase duration, the processor may determine from the evaluation of the recorded data that the user's glucose levels were elevated during the learning phase for the established custom mode (e.g., Karaoke Night Out). Based on the evaluation result (i.e., the determination from the evaluation), the MDA may adjust medicament delivery algorithm parameter settings. Due to the adjusted medicament delivery algorithm parameter settings of the Karaoke Night Out custom mode, the MDA may increase the “aggressiveness” of the algorithm or relax safety constraints (at least in one direction, such as a hyperglycemia direction) of the algorithm, to thereby cause delivery of a larger dosage of medicament such as insulin, as compared to during Normal mode operation, to be output by a wearable drug delivery device.

After completion of the learning phase and the evaluation, the established custom mode, e.g., Karaoke Night Out, is added to the list of other custom modes 116, or rendered selectable such that the user can now select the custom mode/activity without having to go through the initial learning phase or a further portion of the initial learning phase. It should be noted that the established custom mode may be depicted differently on the UI (e.g., in solid black font rather than a partially transparent or a colored font) to indicate that the custom mode has completed the initial learning phase. Whenever the user selects Karaoke Night Out from the list of other custom modes 116, the MDA algorithm may be operable to deliver additional insulin based on the user's increased glucose levels that that have been shown to occur during the activity associated with the Karaoke Night Out custom mode.

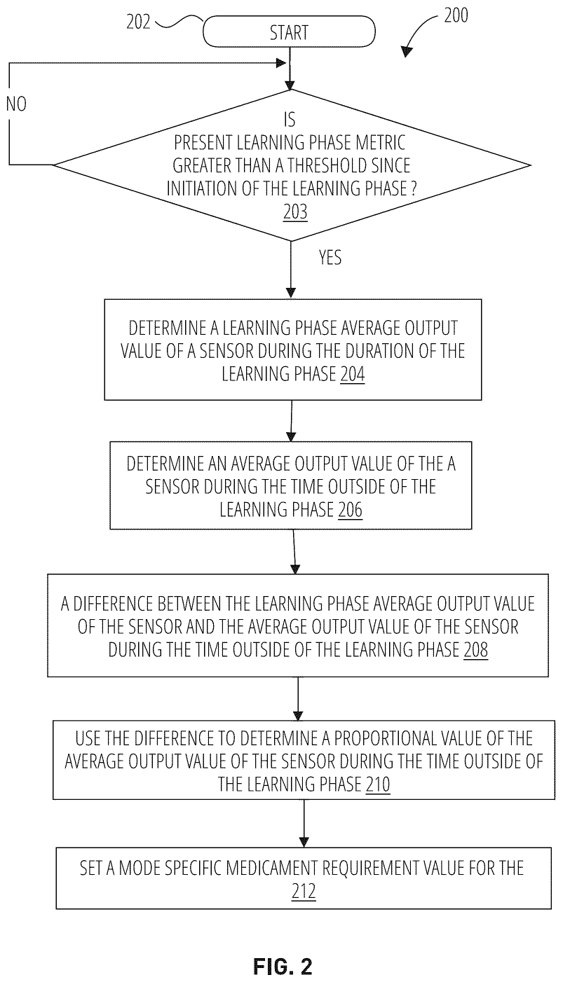

Referring back to FIG. 1A, it may be helpful to describe one or two examples of when the recorded sensor data is evaluated and how the processor generates the list of medicament delivery algorithm parameters. These examples are discussed in more detail with reference to FIG. 2.

In an embodiment, the MDA algorithm may allow users to establish a custom mode of the MDA algorithm, after which the system may automatically determine the appropriate adjustment for the user. This embodiment also allows users to personalize these modes, and even establish modes that provide the user with increased insulin. FIG. 2 illustrates an exemplary custom mode set up process.

The processor may evaluate the recorded sensor data in a number of different ways to determine which medicament delivery algorithm parameters to modify and what values those algorithm parameters should be. The determined medicament delivery algorithm parameters that are to be modified may be placed in a list of medicament delivery algorithm parameters, and be subsequently modified. Alternatively, the determined medicament delivery algorithm parameters may be modified, and then placed in a list of medicament delivery algorithm parameters that include the medicament delivery algorithm parameter settings. However, the appropriate adjustment of the medicament delivery algorithm parameter settings may vary widely depending on the user, their activities, and the intensity of the user's participation in their activities.

As disclosed herein, an MDA may allow users to initiate a custom mode with a personalized name and, in an example, a default or average custom activity mode duration. When this custom mode is first created and activated by the user as described above with reference to FIGS. 1A and 1B, the MDA may not begin the custom mode set up process by initially modifying medicament delivery algorithm parameter settings, but, instead, may begin the custom mode set up process by tagging or annotating sensor data recorded during the learning phase duration. In this manner, non-custom mode algorithm parameter settings may be used during the learning phase to allow the system to determine which algorithm settings need to be adjusted based on sensor values received during the learning phase of the new custom mode activity. Further, the non-custom mode algorithm parameter settings may be adjusted and “trialed” (e.g., adjusted temporarily) during the learning phase to see what impact those adjustments have on the user's sensor values (e.g., glucose measurement values). However, adjustment of the non-custom mode algorithm parameter settings during the learning phase is not required. The recorded sensor data from the learning phase duration may be utilized by the algorithm for future adjustments.

The user interfaces presented during the custom mode set up process may indicate that a learning phase is being implemented when the custom mode is first being established. The learning phase may also be referred to as an “initial learning phase” because it initializes the medicament delivery algorithm parameter settings for the to-be established custom mode, and after the custom mode's unique parameter settings have been established, further “learning phases” may occur when the user engages in the activity so as to further tweak the parameter settings for that particular custom mode. Such further or later learning phases are not required, however, and the user may create a new custom mode for an identical or similar activity, replace a custom mode with a new one, or delete custom modes.

In some examples, the data collected during the learning phase duration is compared to data collected during a period of time that is outside the learning phase duration (i.e., an outside the learning phase time period). For example, the medicament delivery system is configured to collect data nearly continuously from various sensors, such as an analyte sensor that is operable to collect glucose measurements, ketone measurements, or both, and may be further configured to collect data from pedometers, heart rate monitors, blood oxygen sensors, smart accessory devices, and/or the like as described in later examples. Referring back to the example graphical user interfaces of FIG. 1B, the minimum or preferred learning phase duration 121 may be 4 hours as shown in second graphical user interface 110. The time period outside of the learning phase time period may be a time period other than the time period that makes up the learning phase duration (i.e., outside the time when the user has entered or turned on the custom mode). For example, if a user decides to establish a “lunch-time jog” custom mode at 1 pm, with an average or default jogging duration of 1 hour, the minimum or preferred learning phase duration may be 4 hours, and data may be recorded during the time period from 1 μm to 2 pm over 4 successive days, or over the time period when the user has opted to automatically enter the custom “lunch-time jog” mode. The data recorded either before 1 μm or after 2 pm may be data recorded outside the learning phase time period. The data recorded outside the learning phase time period may be used by the medicament delivery algorithm when evaluating the recorded sensor data (i.e., the data recorded during the learning phase duration).

The time period for the established custom mode, such as the “Karaoke Night Out,” may be set by the user to only last for 2 hours (e.g., the user may only participate in karaoke for 2 hours) when the user enters that custom mode again, even though the user set a default or average period of 4 hours when initially setting up the new custom mode. (See, e.g., FIG. 1B, GUI 110). In other words, when entering the “karaoke night out” custom mode in the future, the default duration may be shown in a GUI (e.g., 4 hours), but the user may have the option to change that duration to whatever time period they think they will engage in the custom mode activity at that time (e.g., 2 hours). In any event, the learning phase duration may still need to be 4 hours, as reflected in the example of FIG. 1B, numeral 121, where 4 hours is considered a minimum duration over which to record data in this example. This may alternatively be depicted by a minimum number of analyte readings that need to be received for the learning phase to complete. In the following examples, the learning phase duration is also referred to as the recorded data threshold, and the two terms may be used interchangeably throughout the application.

It is also envisioned that the user can make a setting with the system that automatically engages (i.e., starts) an established custom mode based on a predetermined time, predetermined day, preset date, and/or preset location. For example, the user may attend “Karaoke Night Out” at a specific time(s) on a specific day(s), such as 7 pm on the second Thursday of a month, or the 15th of the month. As such, the user may include a setting that launches the “Karaoke Night Out” custom mode at the particular time (e.g., 7 pm) on the particular day (e.g., Thursday) or particular date. Additionally, or alternatively, the user may use setting that coordinates with a mapping application to set a location setting, for the location of the “Karaoke Night Out.”

In an example, as the time and/or day/date set for the custom mode to start is approaching, the UI can indicate when the upcoming custom mode will turn on; or a pop-up may occur for the user to confirm that they want to enter the custom mode, or simply a reminder like a calendar reminder for an upcoming meeting may be presented.

With regard to a location setting, the system may be configured to cooperate (e.g., receive location information of a user device) with a location determination application (e.g., a mapping application) or global positioning service that is operable to provide a confirmation of the user's location, and the confirmed location can be compared to the location of the “Karaoke Night Out.” Hence, a location application and/or a calendar application of a user device, such as a smartphone, can interact with the MDA to launch respective custom modes based on time of day, date, and/or location.

The user interfaces illustrated herein may be operable to enable a user to set the system to automatically enter into a respective custom mode at a particular time of day, on particular days for a set number of days, particular dates within a month(s), particular locations, or the like. For example, during the creation of the new custom mode as shown in the user interfaces of FIG. 1B, the user may be able to set a particular time, day/date, and/or location for the custom mode to start or be entered. As a result, the user does not have to switch into the respective custom mode because the system enters the respective mode automatically.

In yet another example, the new custom mode may be set to start when other people are nearby, such as spouses, friends, parents, or children. The other people's proximity may be determined by the user's user device using near-field communication or Bluetooth® communication capabilities and the other people's user device.

In an example, the system may be configured to cause the UI to indicate when an upcoming custom mode is set to turn on (e.g., within 1 hour and/or 1 day or 100 feet of the set start time/set start date/set start location of the custom mode). Alternatively, a pop-up notification on a UI may occur requesting a user to confirm that they want to enter/start the custom mode. In another example, the notification may be a reminder, such as an email service reminder, of the upcoming entry/start of the custom mode.

FIG. 2 illustrates an exemplary custom mode set up process. The exemplary custom mode set up process 200 as shown in FIG. 2 may be implemented by a processor (shown in a later example).

The process 200 is an example of a custom mode set up process that may be initiated at Start 202. Initiation may be when the user has selected “Add New button 114” of FIG. 1B, entered a name of the activity or custom mode, or entered the default or average duration 121. This default or average duration 121 may be used to determine how long sensor data should be collected and attributed to the custom mode. For example, if the user enters 1 hour for the new custom mode, sensor data that is collected over the next 1 hour will be tagged or attributed to the new custom mode, and 1 hour of the learning phase will be complete. This process may continue each time the user enters the custom mode (or each time the custom mode is automatically entered, e.g., from 1 p.m.-2 p.m. every day as in the example above) until a recorded data threshold is met. Referring to process 200, upon initiation at Start 202, the processor may execute a time that begins tracking the learning phase duration or recorded data threshold. In the execution of process 200, a processor may store the recorded sensor data in memory, and this data may be retrieved to perform calculations, such as that shown in Equation 1, to evaluate the recorded sensor data collected during the learning phase. For example, the processor may check, at decision block 203, to see if the present learning phase metric (i.e., elapsed time hₖ or a quantity of recorded sensor data points, such as 27 out of 48, or the like) is greater than a threshold (i.e., a learning phase duration, such as 4 hours, represented by h_{th,k}, or a recorded data threshold, which is a threshold number of data points or number of data measurements). The terms “learning phase duration” and “recorded data threshold” may be used interchangeably and both may mean either a period of time, such as 2 hours, 3 hours, 4 hours, or the like, or a number of sensor measurements made during the period of time. For example, if an analyte sensor or similar device provides a glucose measurement value every 5 minutes, in 4 hours, the analyte sensor or similar device provides 48 glucose measurement values, assuming no data values are missed or miscommunicated from the analyte sensor, and this number of analyte measurements may be referred to either as a recorded data threshold or a learning phase duration. In this example, the learning phase duration is a threshold period of time.

If the determination is “No,” at decision block 203, the process 200 re-executes the query in decision block 203, and, when the determination is “No,” sensor data continues to be collected and tagged or attributed to the custom mode activity. During this period, the values of the medicament delivery algorithm parameter settings may remain at their current setting value (i.e., not yet customized for the custom mode activity), or may be “trialed” temporarily, as explained above. However, if the determination is “Yes,” at decision block 203, the process 200 proceeds to step 204. For example, the recorded data threshold may be set to 4 hours of recorded analyte sensor data, or the like, and the processor determines that 4 hours of analyte sensor data has been recorded since the custom mode set up process 200 was initiated.

At optional step 204, the average output value of a sensor during the duration of the learning phase may be determined. Said differently, an average value of the respective recorded sensor data from a particular sensor over the learning phase duration may be determined. As described earlier, the average output value may be referred to as the learning phase average output value. For example, if 48 glucose measurement values (in mg/dL or the like) are received during the learning phase duration, the average of the 48 values may be determined.

At optional step 206, the processor is also operable to determine an average output value of the particular sensor during the time outside the learning phase. This average output value of the sensor during the time outside of the learning phase may be referred to as the outside the learning phase time period average value for the respective sensor. For example, the sensor may be an analyte sensor that provides glucose measurement values and/or ketone measurement values, and/or the like. The recorded sensor data from the learning phase may, for example, be glucose measurement values.

The processor, for example, at optional step 208, is operable to determine a difference between the average sensor data value from the learning phase duration and the outside learning phase time period average value (i.e., an average output value of the sensor during the time outside of the learning phase). The difference is an indication of how the user's analyte metabolism reacts to the activity of the custom mode.

At 210, the difference may be used to determine a proportional value of the average output value of the sensor during the time outside of the learning phase. In a glucose measurement value example, if the learning phase average output value were to be 180 mg/dL and the outside the learning phase time period average value was 150 mg/dL, the difference would be 30 mg/dL, and the proportional value determined at step 210 may be 30 mg/dL divided by 150 mg/dL, which equals 0.20 or 20%.

Using the proportional value of 20% from the example, the processor, at 212 may set a mode specific medicament requirement. This may be done by adding the proportional value (e.g., 0.20) to 1, which results in a mode specific medicament requirement M_{i,k} of 1.20. The mode specific medicament requirement may be used to modify one or more medicament delivery algorithm parameter settings. For example, a medicament delivery algorithm parameter setting in the Normal mode may be a nominal value of 100 and based on the example from the discussion of step 210, the medicament delivery algorithm parameter setting value of 100 may be, for example, multiplied by the mode specific medicament requirement value 1.20, which would result in a modified medicament delivery algorithm parameter setting value of 120.00. Alternatively, the medicament delivery algorithm parameter setting value of 100 may be, for example, divided by 1.20, which would result in a modified medicament delivery algorithm parameter setting value of 83.33. In other words, one or more medicament delivery algorithm parameter settings may be adjusted proportionally to the difference between the average sensor data value from the learning phase duration and the outside learning phase time period average value.

Equation 1 below illustrates an exemplary mathematical implementation of the above process 200.

In an example, during the custom mode set up process (as shown in FIGS. 1A, 1B and 2), the MDA algorithm may compare the glucose control outcomes during this period versus outside of this period under the same insulin delivery conditions to determine a mode specific medicament requirement value, represented by M_{i,k}. In one embodiment, this may be represented by the following function M_{i,k} that utilizes Equation 1:

$${M}_{i , k} = \{ \begin{matrix}{h}_{k} > {h}_{th , k} & 1 + \frac{( \frac{{\sum }_{j = 1}^{12 \cdot {h}_{k}} CGM ( i + j )}{12 \cdot {h}_{k}} - \frac{{\sum }_{j = 1}^{12 \cdot {h}_{o}} CGM ( i - j )}{12 \cdot {h}_{o}} )}{\frac{{\sum }_{j = 1}^{12 \cdot {h}_{o}} CGM ( i - j )}{12 \cdot {h}_{o}}} \\ {h}_{k} \leq {h}_{th , k} & 1\end{matrix} . \qquad ( Equation 1 )$$

In this example, the first term in the numerator provides the processor with an average glucose measurement value (from, for example, a CGM) during that activity (e.g., activity k). In Equation 1, the first term is a sum of all CGM values during the learning phase duration of the custom mode, divided by number of hours the user was in that activity, where the number of hours is represented by a CGM providing a value 12 times or cycles per hour (cycles per hour may vary depending on the device and settings). The second term in the numerator provides the processor with the average CGM value when the user is not in the custom mode (i.e., an average value from outside the learning phase time period, which may be, for example, 2 hours, 6 hours, 8 hours, 20 hours, or the like). In one exemplary implementation, this “outside the learning phase timer periods” may be immediately before the learning phase time period, immediately after the learning phase time period, or may be before and after the learning phase time period The term h_{th,k} is a threshold value for the number of hours of the activity (e.g., 4 hours). The term h_{th,k} is also the learning phase duration or recorded data threshold (in hours). In an embodiment, the processor may limit taking action (e.g., modifying medicament delivery algorithm parameter settings) until crossing the threshold value h_{th,k}; or the processor might limit action taken based on amount of data per the hₖ/hₜₕ ratio.

Continuing with the glucose measurement values as the recorded sensor data in this example, the user's mean glucose concentration for the overall duration of data available for the k^{th} custom mode hours hₖ may be compared against the mean glucose for all available data outside of the current mode hₒ, if the amount of recorded data greater than h_{th,k} (i.e., the amount of recorded data exceeds the recorded data threshold) is available for establishing the custom mode. As mentioned in earlier examples, sufficient data may be considered by the processor to be at least 4 hours of or a set number of data points (i.e., number of glucose measurement values) of recorded sensor data, where the learning phase duration is also 4 hours. In some examples, a 4-hr learning phase duration defines an amount of recorded sensor data sufficient to determine medicament delivery algorithm parameter settings for the respective custom mode.

In an operational example of how the mode specific medicament requirement M_{i,k} determined from Equation I above, a mean CGM value for a user during an activity being established may be 140 mg/dL, and a user's mean CGM value outside of the activity may be 150 mg/dL, and the number of custom mode hours hₖ may be less than the learning phase duration h_{th,k}. Thus, when hₖ is less than h_{th,k}, the numerator may have values (140 mg/dL-150 mg/dL), which is divided by 150 mg/dL. Numerically, the numerator and denominator are, respectively, −10/150, which equals (−) 0.0667. The quotient −0.0667 is the value within the large parenthesis of Equation 1. Equation 1 has a value of 1+ (−)0.0667, which equals 0.9333. Hence, M_{i,k} in this example is 0.9333 because the current learning phase time hₖ is less than a learning phase duration threshold h_{th,k}. It should be noted again that this is just one specific exemplary implementation.

In another example, the processor, when executing the MDA is configured to incrementally make preliminary medicament delivery algorithm parameter settings when less than the entire recorded data threshold is obtained due to less than the entire learning phase duration passing.

In such an instance, the proportional value of the average output value of the sensor during the time outside of the learning phase may be determined as in step 210 of FIG. 2. However, the mode specific medicament requirement M_{i,k} may be determined by other methods. Alternatively, the level of adjustment due to the mode specific medicament requirement may be a linear conversion to this threshold based on the duration of available data, with a 100% conversion after a threshold (e.g., recorded data threshold or learning phase duration threshold h_{th,k}), as follows:

$${M}_{i , k} = 1 + \frac{{h}_{k}}{{h}_{th , k}} ( \frac{( \frac{{\sum }_{j = 1}^{12 \cdot {h}_{k}} CGM ( i + j )}{12 \cdot {h}_{k}} - \frac{{\sum }_{j = 1}^{12 \cdot {h}_{o}} CGM ( i - j )}{12 \cdot {h}_{o}} )}{\frac{{\sum }_{j = 1}^{12 \cdot {h}_{o}} CGM ( i - j )}{12 \cdot {h}_{o}}} ) \qquad ( Equation 2 )$$

In this example, if the amount of data is minimal due to the time for data collection, e.g., hₖ, is less than the learning phase duration threshold, h_{th,k}. the mode specific medicament requirement M_{i,k} may be set proportionally. For example, the time for data collection hₖ may only be 1 hour while the learning phase duration threshold h_{th,k} is 4 hours, the proportional value may be reduced further by the fractional value of hₖ/h_{th,k}. Using the average numbers from the example above, the learning phase average output value may be 180 mg/dL and the outside the learning phase time period average value may be 150 mg/dL. The difference is 30 mg/dL, and the proportional value remains 0.20. However, due to the minimal amount of data, the 0.2 may be further reduced by the fractional value hₖ/h_{th,k}. Using the values of 1 hour for hₖ and 4 hours for h_{th,k}, the fractional value is ¼ or 0.25. Thus, the proportional value (0.20) is reduced by 25% to 0.05. Hence, the mode specific medicament requirement is equal to 1.05 when only 1 hour of data is collected.

As more data is collected, the fractional value gets closer to 1 or 100% and eventually, the mode specific medicament requirement M_{i,k} is based on the full value (0.20) of the proportional value. In an example, the full value of the proportional value is the calculated amount of the proportional value not reduced by a fractional value. This example allows for a dynamic generation of the mode specific medicament requirement that is applied to the medicament delivery algorithm parameter settings. In an example, the mode specific medicament requirement may be calculated every hour, every half-hour, every quarter-hour, or the like until the learning phase duration threshold h_{th,k} is satisfied. Note that the learning phase duration threshold h_{th,k} may also be referred to as the recorded data threshold.

In an operational example of Equation 2 for determining a value of the mode specific medicament requirement M_{i,k}, a mean CGM value for a user during an activity being established may be 140 mg/dL, and a user's mean CGM value outside of the activity may be 150 mg/dL, and that hₖ is equal to h_{th,k}. Thus: (140 mg/dL-150 mg/dL) divided by 150 mg/dL, which numerically is −10/150=−0.0667. The value −0.0667 is the value within the large parenthesis of Equation 2. Equation 2 has a value of 1+ (−)0.0667, which equals 0.9333. Hence, the mode specific medicament requirement M_{i,k}, in this example, is 0.9333.

The example mode specific medicament requirement value determined according to Equation 2 may be used to modify insulin delivery when the number of hours of recorded sensor data is less than the number of hours needed to meet the recorded data threshold. The amount of insulin delivered may be adjusted using the current amount, or latest amount of recorded sensor data, where the amount of insulin delivered is scaled based on the relative duration of available history to the value of the duration threshold (hₖ/h_{th,k}).

In yet another example, the mode specific medicament requirement may be calculated based on stages of participation in the custom mode. In this example, a finalized mode specific medicament requirement M_{f} may be determined over a course of a day, or the like, as the user enters and re-enters the specific custom mode. In a specific example, the mode specific medicament requirement M_{f} may be updated whenever a user enters a particular custom mode, such as Karaoke Night Out. The adjustment of the mode specific medicament requirement may also be updated with each activation of the particular custom mode, based on a moving average of the previous instances when the users were in the same custom mode. For example, the adjustment to the mode specific medicament requirement may be made with a weighted average based on the duration of the user's most recent selection dᵢ, such as the following:

$${M}_{f , k} = ( 1 - \frac{{d}_{i}}{D} ) {M}_{f , k - 1} + \frac{{d}_{i}}{D} {M}_{i , k} \qquad ( Equation 3 )$$

In Equation 3, the variable D may be a tunable parameter to determine the speed of the update. A reasonable value for the parameter D may be 24, indicating 24 hours of new data may lead to a 100% weight on the mode specific medicament requirement from the new data. The variable dᵢ as mentioned above is the duration of the user's i^{th} most recent previous selection, and adjusts the most recent estimate M_{f,k-1} based on the new medicament value during that selection.

Of course, other methods may also be utilized to calculate the mode specific medicament requirement. For example, the processor may be configured to compare the total insulin delivery values for the learning phase duration to the total insulin delivery values for the outside the learning phase time period. In another example, the recorded sensor data from the learning phase may be data that when evaluated indicates either a hyperglycemic incident or a hypoglycemic incident. The processor may count the number of either hyperglycemic incidents or hypoglycemic incidents, and compare the number of either hyperglycemic incidents or hypoglycemic incidents that occurred during the learning phase duration to the number of hyperglycemic incidents or hypoglycemic incidents that occurred during the outside the learning phase time period.

Once calculated, the MDA may utilize the mode specific medicament requirement to adjust a wide range of algorithm parameters, such as input parameters and/or constraints, when the user activates the particular custom mode. For example, an input TDI (or total daily insulin), insulin-on-board or IOB may be adjusted, a duration of insulin action (DIA) may be adjusted, a target glucose setpoint, a maximum medicament delivery, an insulin sensitivity, a correction factor, or other parameters, may be adjusted. By way of particular example for some of these parameters, the mode specific medicament requirement adjustments M_{i,k} may be applied as follows:

TABLE 1

| | Incorporation of mode specific insulin |
|---|---|
| MDA Parameter | sensitivity |
| Input TDI | TDit = min(1.1, max(0.9, Mi,k)) · TDI |
| IOB constraint | $I O {B}_{f} = \frac{I O B}{min ( 1.1 , max ( 0.9 , {M}_{i , k} ) )}$ |
| Target Glucose Concentration | SP_{f} = min(SPₘₐₓ, min(SPₘᵢₙ, SP − (M_{i,k} − 1) · (SPₘₐₓ − SPₘᵢₙ))) |
| Maximum insulin delivery limit | UB_{f} = min (1.1, max (0.9, (M_{i,k})² ) UB |
| (upper bound constraint) | |

In Table 1 above, M_{i,k} is the mode specific medicament requirement value and may be used by the MDA when the user selects a respective mode. Each mode may have its own mode specific medicament requirement value, or alternatively, similar modes, such as cycling and swimming, may have separate mode specific medicament requirement values. In some instances when each mode has a separate mode specific medicament requirement value, the actual value of the mode specific medicament requirement value may be the same value. Of course, the actual value of the mode specific medicament requirement value may be a different value for one or more activities, or each activity may have a different mode specific medicament requirement value.

The different MDA parameters mentioned above may be used in various determinations of medicament delivery dosages, e.g., IOB values or durations may be adjusted which would directly impact medicament delivery dosage calculations, or maximum medicament delivery limits, such as the listed upper bound constraint or maximum insulin delivery limit, or other MDA parameters may be adjusted based on the mode specific medicament requirement M_{i,k} value, which would directly impact medicament delivery dosage calculations as would be understood by one of ordinary skill in the art.

In an example, the mode specific medicament requirement M_{i,k} value may be calculated at various time points as explained above with respect to Equation 2. Various MDA parameters may be adjusted based on the value of the mode specific medicament requirement M_{i,k}. In some examples, MDA parameters may be adjusted continuously and/or temporarily (e.g., to trial different MDA parameters) as the learning phase time progresses (e.g., as the value of h_{i,k} increases). Alternatively, MDA parameters may be adjusted after the learning phase time matches or exceeds the learning phase duration. In yet another alternative, MDA parameters may continue to be adjusted whenever the custom mode is activated. Of course, combinations of these alternative adjustment examples may be implemented for the MDA parameters.

FIG. 3A illustrates an exemplary step in a process of selecting a preset activity in accordance with the disclosed subject matter. The user device 300 includes touchscreen display, for example, on which a user interface 302 may present data related to a user's AMD system. The user interface 302 is presented, for example, when a user is monitoring their glucose status, such as a user's most recent glucose value (e.g., 121 mg/dL), glucose value trend (e.g., arrow adjacent most recent glucose value that points either up/down/right based on whether the glucose value is trending upward/downward or steady, operating mode (e.g., automated, manual, custom mode, closed-loop, open-loop, hybrid mode, or the like), an amount of medicament on board (e.g., insulin on board (IOB) in Units), an amount of medicament delivered in a last bolus dosage (in Units), when the last bolus was delivered (e.g, date and time), a chart plotting past measurement values, or the like. Additionally, the user interface 302 may present a mode icon 301, which may show whether a custom mode is active or inactive based, for example, on color (e.g., a gray icon may indicate that custom mode settings are inactive; a dotted icon or a yellow color may indicate that the custom mode is still in the learning phase; a black or green icon may indicate that the custom mode is active). The mode icon 301 may take other various forms than color to indicate its function, such as a word, a specific symbol, or the like. In addition, the MDA may be operable to cause the presentation of an audio indication of the mode or cause a wireless signal, such as Bluetooth or the like, to a smart accessory device or the like to be output.

The user interface 302 may also show a status of other components of the AMD system, such as the wearable medicament delivery device, a bolus medicament delivery request button, and additional menu items. The user interface 302 may present the initial screen for beginning the process for selecting a preset activity mode, or custom mode.

An example operational process may begin with selection of the mode icon 301 to activate (or inactivate) a custom mode.

FIG. 3B illustrates an exemplary step in a process of selecting a preset activity following the step in FIG. 3A in accordance with the disclosed subject matter.

In an operational example, assume mode icon 301 shows that any custom mode is inactive; then after a user selects the mode icon 301, the user device 300 may stop presenting user interface 302 and begin presenting user interface 304. The user interface 304 may be configured to present a menu of “Activity Presets,” which are the custom modes established by the user. For purposes of discussion, the activity presets presented in user interface 304 include only custom modes established by the custom mode set up processes described herein. But, in other examples, the Activity Presets may include system default Activity Presets that may be settings that are generic to most users as opposed to the custom modes established by the processes described herein. Further, there may be an option to set up a new custom mode or activity preset in this UI. Still further, custom modes or activity presets that have not completed the initial learning phase may also be depicted, though perhaps in a different format (e.g., grey text or slightly transparent text), as explained earlier.

When a user wishes to begin one of the custom modes, the user selects an activity preset from the Activity Presets Menu presented in the user interface 302. In the example, the user may wish to go biking, and may choose selected activity preset 303, which has medicament delivery algorithm parameter settings customized for when the user is biking (based on a prior setup on an initial learning phase for this particular custom mode).

FIG. 3C illustrates another exemplary step in a process of selecting a preset activity in accordance with the disclosed subject matter.

After making a selection in user interface 304, such as selecting the biking activity shown as the selected activity preset 303, the user may be presented with a user interface, such as 305, that enables review of the selected activity preset 303. The user interface 305 may present an activity review screen for biking, which may include an activate activity button 310 and an edit activity button 306. By selecting the edit activity button 306, the user may be given an opportunity to change one or more of activity settings 311, which may include medicament delivery algorithm parameter settings. For example, the activity settings 311 show target glucose measurement value (shown as Target BG), correction weight and average duration (e.g., 4 hours) of the activity. The user may update the duration for which they desire to engage in the activity. For example, the user may change the “average duration” or “default duration” from 4 hours to 1 hour. This may change the duration of the particular custom activity only once, or it may update the default setting such that the next time the user chooses this custom mode or activity preset, the new value will appear (e.g., 1 hour instead of 4 hours). The user may also be enabled to modify a target BG or a correction weight; alternatively, these settings may be made unalterable by the user, particularly if they were The target glucose measurement value and the correction weight are medicament delivery algorithm parameter settings that may be determined by the system during the learning phase duration (e.g., based on average CGM measurements during the learning phase duration and calculation of a mode specific medicament requirement value). Other algorithm parameter settings may also be determined, as discussed above, and may also be presented here for the user's information or to allow the user to modify them.

Alternatively, or after making edits, the user can select activate activity button 310 to enter the custom mode or begin utilizing the activity preset. Upon activating the selected mode, the user device 300 may transition from presenting user interface 305 to presenting user interface 309.

FIG. 3D illustrates another exemplary presentation of a selected preset activity user interface in accordance with the disclosed subject matter. In FIG. 3D, the user interface 309 includes an edit activity button 306, an activated mode icon 307, a custom mode glucose target 308 (as modified for this particular custom mode, as discussed in examples above), and a target blood glucose line 312 based on the custom mode glucose target.

As shown by the change in appearance of the inactive mode icon 301 of FIG. 3A to the activated mode icon 307 in user interface 309, the activated mode icon 307 indicates that the biking mode activity has been activated. Alternatively, or additionally as shown, the presentation of “Biking Mode” at the top of the user interface 309 also indicates that the custom mode or preset activity “Biking Mode” has been activated. Whatever the selected activity preset 303, the current mode may be shown by the activated mode icon 307 in the right hand corner of the user interface 309. The activated mode icon 307 may be shown in a color different from the inactive mode icon 301 to show the selected activity preset 303 (or custom mode) is activated. In an example, the “Biking Mode” user interface presents similar categories of information as the “Automated Mode” presented in user interface 302, such as a user's most recent glucose value (e.g., 121 mg/dL), glucose value trend (e.g., arrow adjacent most recent glucose value that points either up/down/right based on whether the glucose value is upward/downward or steady, operating mode (e.g., Biking mode), an amount of medicament on board (e.g., insulin on board (IOB) in Units), an amount of medicament delivered in a last bolus dosage (in Units), when the last bolus was delivered (e.g, date and time), and a chart plotting past measurement values. In one example, the chart may indicate when a custom user mode was entered and the name of that custom user mode. For example, a portion of the chart may be highlighted, or a horizontal line may be added indicating when a custom mode started and ended.

Recall that in FIG. 3C the Target blood glucose was 140 mg/dL, which is indicated in user interface 309 as the mode glucose target 308 and target blood glucose line 312 in FIG. 3D.

FIG. 4 illustrates an exemplary medicament delivery system operable to implement the examples disclosed herein.

The medicament system 400 is suitable for delivering a liquid medicament, such as insulin to a user as well as monitor and evaluate senor measurement values in accordance with the disclosed embodiments. The medicament system 400 may include a wearable medicament device 402, a controller 404 and an analyte sensor(s) 406.

The medicament delivery system 400 may include a wearable medicament delivery device 402, a controller 404, an analyte sensor(s) 406, a housing(s) 408, a cloud-based services 410, a network 412, a computing device 114, a fitness device 416, a smart accessory device 418, another wearable device 120, a communication circuitry 422, a transceiver 424, a transceiver 426, a user interface 428, a processor 430, a memory 432, other data 434, a control application 436, a settings 438, a communication link 140, a memory 442, a settings 444, a control application 446, other data 448, a processor 450, a user interface 452, a pump 454, a reservoir 456, a communication circuitry 158, a continuous glucose monitor 460a, a continuous glucose monitor 460b, a ketone sensor 162a, a ketone sensor 462b, a communication links 464, a communication link 466, and a communication link 468.

The wearable medicament delivery device 402 may be a wearable device that is worn on the body of the user. The wearable medicament delivery device 402 may be directly coupled to a user (e.g., directly attached to a body part and/or skin of the user via an adhesive, or the like). In an example, a surface of the wearable medicament delivery device 402 may include an adhesive to facilitate attachment to the skin of a user.

The wearable medicament delivery device 402 may include a processor 450. The processor 450 may be implemented in hardware, software, or any combination thereof. The processor 450 may, for example, be a microprocessor, a logic circuit, a field programmable gate array (FPGA), an application specific integrated circuit (ASIC) or a microprocessor coupled to a memory. The processor 450 may maintain a date and time as well as be operable to perform other functions (e.g., calculations or the like). The processor 450 may be operable to execute a control application 446 stored in the memory 442 that enables the processor 450 to direct operation of the wearable medicament delivery device 402. The control application 446 may control insulin delivery to the user per an MDA control approach as describe herein. For example, the control application 446 may be an MDA. The memory 442 may hold settings 444 for a user, such as MDA algorithm settings, such as maximum insulin delivery, insulin sensitivity settings, total daily insulin (TDI) settings and the like. The memory may also store other data 448, such as ketone values, total daily insulin values, glucose measurement values from analyte sensor(s) 406 or controller 404, insulin dosage amounts (both basal and bolus) and the like from previous minutes, hours, days, weeks, or months.

The analyte sensor(s) 406 may be operable to collect physiological condition data, such as ketone values, ketone values with a time stamp, glucose measurement values (also referred to herein as “blood glucose values” or “blood glucose”), glucose measurement values and a timestamp, and the like. In some examples, the ketone values and/or the glucose values are shared with the wearable medicament device 402, the controller 404, or both. In an additional example, the analyte sensor(s) 406 may include multiple sensors, such as a continuous glucose monitor 460a and a ketone sensor 462a. In a further example, the wearable medicament delivery device 402 may optionally include a continuous glucose monitor 460b and a ketone sensor 462b, which may be removably incorporated or fully integrated within the wearable medicament delivery device 402. For example, the continuous glucose monitor 460b and the ketone sensor 462b may be incorporated in one or more housing(s) 408 of the wearable medicament delivery device 402. Note that ketones may also be detected using a breath sensor (which is not shown but may be incorporated in the controller 404) or urine content sensor and stored in the respective memories 442 and 432 via a user input of the ketone levels; however, a subcutaneous ketone sensor(s) gives more accurate information and is more continuous. As described herein, the MDA and automated medicament delivery (AMD) system is described based on receiving ketone values received subcutaneously. Use of a ketone breath sensor or urine sensor as part of or in addition to the analyte sensor(s) 406 may delay receipt of the ketone values and modifications to the system 400 may be made.

For example, the communication circuitry 458 of the wearable medicament delivery device 402 may be operable to communicate with the analyte sensor(s) 406 and the controller 404 as well as the devices: fitness device 416, smart accessory device 418, and other wearable device 420 (e.g., GPS watch or the like). The communication circuitry 458 may be operable to communicate via Bluetooth, cellular communication, near field communication (NFC) and/or other wireless protocols. While not shown, the memory 442 may include both primary memory and secondary memory. The memory 442 may include random access memory (RAM), read only memory (ROM), optical storage, magnetic storage, removable storage media, solid state storage or the like.

The wearable medicament delivery device 402 may include a reservoir 456. The reservoir 456 may be operable to store liquid drugs, medicaments, or therapeutic agents suitable for automated delivery, such as, insulin, glucagon-like peptide-1 (GLP-1) receptor agonist, pramlintide, glucose-dependent insulintropic polypeptide (GIP), other hormones, or co-formulations of two or more of glucagon, GLP-1, pramlintide, and insulin; as well as pain relief drugs, such as opioids or narcotics (e.g., morphine, or the like), methadone, blood pressure medicines, chemotherapy drugs, weight loss medicaments or supplements, fertility drugs, or the like.

A fluid path to the user may be provided via tubing and a needle/cannula (not shown). The fluid path may, for example, include tubing coupling the wearable medicament delivery device 402 to the user (e.g., via tubing coupling a needle or cannula to the reservoir 456). The wearable medicament delivery device 402 may be operable based on control signals from the processor 450 to expel the liquid drugs, medicaments, or therapeutic agents, such as insulin, from the reservoir 456 to deliver doses of the drugs, medicaments, or therapeutic agents, such as the insulin, to the user via the fluid path. The processor 450 may be operable to cause insulin to be expelled from the reservoir 456.

There may be one or more communication links 464 with one or more devices physically separated from the wearable medicament delivery device 402 including, for example, a controller 404 of the user and/or a caregiver of the user and/or an analyte sensor(s) 406. The communication links 464 may include any wired or wireless communication link operating according to any known communications protocol or standard, such as Bluetooth®, NFC, Wi-Fi, a near-field communication standard, a cellular standard, or any other wireless protocol. The analyte sensor(s) 406 may communicate with the wearable medicament delivery device 402 via a wireless communication link 440 and/or may communicate with the controller 404 via a wireless communication link 466.

The wearable medicament delivery device 402 may also include a user interface 452, such as an integrated display device for displaying information to the user, and in some embodiments, receiving information from the user. For example, the user interface 452 may include a touchscreen and/or one or more input devices, such as buttons, knob(s), or a keyboard that enable a user to provide an input.

In addition, the processor 450 may be operable to receive data or information from the analyte sensor(s) 406 as well as other devices that may be operable to communicate with the wearable medicament delivery device 402.

The wearable medicament device 402 and/or the controller 404 may interface with a network 412. The network 412 may include a local area network (LAN), a wide area network (WAN) or a combination therein. A computing device 414 may be interfaced with the network, and the computing device may communicate with the wearable medicament delivery device 402. The computing device 414 may be a healthcare provider device through which a user's controller 404 may interact to exchange information, store settings, and the like. The AMD algorithm operating, as or in cooperation with, the control application 436 may present a graphical user interface on the computing device 414 enabling the input and presentation of information related to the AMD algorithm and the example techniques and processes described herein.

As mentioned, the medicament system 400 may include an analyte sensor(s) 406 for sensing the levels of one or more analytes of a user. The analyte levels may be used as physiological condition data and be sent to the controller 404 and/or the wearable medicament delivery device 402. The analyte sensor(s) 406 may be coupled to the user by, for example, adhesive or the like and may provide information or data on one or more medical conditions and/or physical attributes of the user. The analyte sensor(s) 406 may be a continuous glucose monitor (CGM), ketone sensor, or another type of device or sensor(s) that provides glucose measurements and/or ketone measurements. The analyte sensor(s) 406 may be physically separate from the wearable medicament delivery device 402 or may be an integrated component thereof. The analyte sensor(s) 406 may provide the processor 430 and/or processor 450 with physiological condition data indicative of measured or detected glucose levels of the user. The information or data provided by the analyte sensor(s) 406 may be used to modify a medicament delivery schedule and thereby cause the adjustment of medicament operations of the wearable medicament delivery device 402.

The controller 404, in more detail, may include a processor 430 and a memory 432. The controller 404 may be a special purpose device, such as a dedicated personal diabetes manager (PDM) device. The controller 404 may be a programmed general-purpose device that is a portable electronic device, such as any portable electronic device, smartphone, smartwatch, fitness device, tablet or the like including, for example, a dedicated processor, such as processor, a micro-processor, or the like. The controller 404 may be used to program or adjust operation of the wearable medicament delivery device 402 and/or the analyte sensor(s) 406. The processor 430 may execute processes to control the delivery of the medicament, drug, or therapeutic agent to the user for the purpose of managing a user's blood glucose and/or ketone levels. The processor 430 may also be operable to execute programming code stored in the memory 432. For example, the memory 432 may be operable to store a control application 436, such as an AMD algorithm for execution by the processor 430. The control application 436 may be responsible for controlling the wearable medicament delivery device 402, including the automated delivery of medicament, a drug, or therapeutic agent based on recommendations and instructions from the AMD algorithm, such as those recommendations and instructions described herein.

The memory 432 may store one or more applications, such as control application 436, and settings 438 for the wearable medicament delivery device 402 like those described above. In addition, the memory 432 may be operable to store other data 434 and/or computer programs, such as medicament history, glucose measurement values over a period of time, total daily insulin values, ketone values, and the like. For example, the memory 432 is coupled to the processor 430 and operable to store programming instructions, such as the control application 436 and settings 438, and data, such as other data 434, related to a glucose of a user and/or data related to an amount of insulin expelled by the wearable medicament delivery device 402.

The controller 404 may include a user interface (UI) 428 for communicating with the user. The user interface 428 may include a display, such as a touchscreen, for displaying information. The touchscreen may also be used to receive input when it is a touch screen. The user interface 428 may also include input elements, such as a keyboard, button, knob, or the like. In an operational example, the user interface 428 may include a touchscreen display (including a display and user input circuitry, such as touch sensitive circuits and the like) controllable by the processor 430 and be operable to present a graphical user interface and receive inputs via the user input circuitry, the touchscreen display may be operable to generate a signal indicative of the respective diet types, number of days, diet cycles, keto days, sick day mode, sickness symptoms, intermittent fasting mode, fasting mode, religious fasting mode, location information, calendar information, or the like. The touchscreen display, under control of the processor 430, may be operable to receive inputs such as those described with reference to the examples of FIGS. 1-3D. The graphical user interfaces discussed herein with respect to the examples may be generated by the processor 430 of the controller 404 and be presented on the UI 428.

The controller 404 may interface via a wireless communication link of the wireless communication links 464 with a network, such as a LAN or WAN or combination of such networks that provides one or more servers or cloud-based services 410 via communication circuitry 422. The communication circuitry 422, which may include transceivers 424 and 426, may be coupled to the processor 430. The communication circuitry 422 may be operable to transmit communication signals (e.g., command and control signals) to and receive communication signals (e.g., via transceivers 424 or 426) from the wearable medicament delivery device 402 and the analyte sensor(s) 406. In an example, the communication circuitry 422 may include a first transceiver, such as 424, that may be a Bluetooth transceiver, which is operable to communicate with the communication circuitry 422 of the wearable medicament delivery device 402, and a second transceiver, such as 426, that may be a cellular or Wi-Fi transceiver operable to communicate via the network 412 with computing device 414 or with cloud-based services 410.

The cloud-based services 410 may be operable to receive and store user history information, such as glucose measurement values over a set period of time (e.g., days, months, years), a medicament history that includes insulin delivery amounts (both basal and bolus dosages) and insulin delivery times, types of insulin delivered, indicated meal times, glucose measurement value trends or excursions or other user-related diabetes treatment information, specific factor settings including default settings, present settings and past settings, or the like.

Other devices, like smart accessory device 418 (e.g., a smartwatch or the like), fitness device 416 and other wearable device 420 may be part of the medicament system 400. These devices may communicate with the wearable medicament delivery device 402 to receive information and/or issue commands to the wearable medicament delivery device 402. These devices 416, 418, and 420 may execute computer programming instructions to perform some the control functions otherwise performed by processor 450 or processor 430. These devices 416, 418, and 420 may include user interfaces, such as touchscreen displays for displaying information such as current glucose level, medicament on board such as insulin on board, medicament history, or other parameters or treatment-related information and/or receiving inputs, such as those described with reference to the examples of FIGS. 1-3D. The display may, for example, be operable to present a graphical user interface for providing input, such as request a change in basal insulin dosage or delivery of a bolus of insulin. These devices 416, 418, and 420 may also have wireless communication connections with the analyte sensor(s) 406 to directly receive glucose level data or receive in parallel a presentation of the graphical user interface as shown in FIGS. 1B and 3A-3D.

In another operational example, the controller 404 may be operable to execute programming code that causes the processor 430 of the controller 404 to perform the following functions. The processor 430 of the controller 404 may execute an AMD algorithm via control application 436 stored in the memory 432. The processor may be operable to present graphical user interfaces as described herein on a user interface that is at least one component of the user interface 428 and establish custom modes as described herein with reference to the examples of FIGS. 1-3D. For example, the user interface 428 may be a touchscreen display controlled by the processor 430, and the user interface 428 is operable to present a graphical user interface that enables establishing a custom mode as described herein.

The processor 430 is also operable to collect physiological condition data related to the user from sensors, such as the analyte sensor(s) 406 or heart rate data, for example, from the fitness device 416 or the smart accessory device 418. In an example, the processor 430 executing the AMD algorithm may determine a dosage of medicament to be delivered based on the collected physiological condition of the user and/or a specific factor determined based on the subjective medicament need parameter. The processor 430 may output a control signal via one of the transceivers 424 or 426 to the wearable medicament delivery device 402. The outputted signal may cause the processor 450 to deliver command signals to the pump 454 to deliver an amount of the determined dosage of medicament in the reservoir 456 to the user based on an output of the AMD algorithm. The processor 450 may also be operable to perform calculations to update or establish settings of the AMD algorithm as discussed as herein. Modifications to the AMD algorithm settings may be stored in the memory 432, for example, as settings 438.

While the system 400 is described with reference to delivery of insulin and the use of an AMD algorithm, the system 400 may be operable to implement a medicament regimen via a medication delivery algorithm using a number of different liquid or therapeutic drugs/medicaments, such as those described above with reference to the reservoir 456.

Software related implementations of the techniques described herein, such as the examples described with reference to FIGS. 1-16 may include, but are not limited to, firmware, application specific software, applet, or any other type of computer readable instructions that may be executed by one or more processors. The computer readable instructions may be provided via non-transitory computer-readable media. The various elements of the processes described with reference to the figures may be implemented in devices, apparatuses or systems that may include various hardware elements, software elements, or a combination of both. Examples of hardware elements that may be the basis for a computer processor may include structural members, logic devices, components, processors, microprocessors, circuits, processors, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements may include software components, programs, applications, computer programs, application programs, system programs, software development programs, machine programs, operating system software, middleware, firmware, software modules, routines, subroutines, functions, processes, procedures, software interfaces, application program interfaces (API), instruction sets, computing code, computer code, code segments, computer code segments, words, values, symbols, or any combination thereof.

Some examples of the disclosed device or processes may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein. The present disclosure furthermore relates to computer programs comprising instructions (also referred to as computer programming instructions) to perform the aforementioned functionalities. The instructions may be executed by a processor. The instructions may also be performed by a plurality of processors for example in a distributed computer system. The computer programs of the present disclosure may be for example preinstalled on, or downloaded to the medicament delivery device, management device, fluid delivery device, e.g. their storage.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined solely by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, novel subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible considering this disclosure. It is intended that the scope of the present disclosure be limited not by this detailed description, but rather by the claims appended hereto. Future filed applications claiming priority to this application may claim the disclosed subject matter in a different manner and may generally include any set of one or more features as variously disclosed or otherwise demonstrated herein.

What is claimed is:

1. A device for establishing a customized mode of a medicament delivery algorithm, the device configured to:
receive an indication to establish a custom mode;
implement an initial learning phase, wherein a processor executing the initial learning phase receives and evaluates sensor data for a duration of time; and
based on a result of the initial learning phase, set medicament delivery algorithm parameters for the custom mode.

2. The device of claim 1, wherein the duration of time is a default duration of time or a duration of time received via an input device.

3. The device of claim 1, further configured to:
give a customized mode name to the custom mode; and
store the set medicament delivery algorithm parameters in association with the given mode name.

4. The device of claim 1, wherein when implementing the initial learning phase, the device is configured to:
receive an indication of a duration of the initial learning; and
replace the default duration of time with the received indication of the duration of the initial learning.

5. The device of claim 1, wherein when executing the learning phase, the device is configured to:
record sensor data provided by one or more sensors related to an activity covered by the custom mode;
evaluate recorded sensor data to establish parameter settings of the activity mode; and
generate a list of medicament delivery algorithm parameters and recommended settings for each parameter in the list of medicament delivery algorithm parameters.

6. The device of claim 5, wherein the device is further configured to:
determine whether the recorded sensor data is sufficient to recommend a setting value for each parameter in the list of medicament delivery algorithm parameters.

7. The device of claim 5, wherein the processor is further configured to:
determine whether the recorded sensor data is longer than a recorded data threshold; and
in response to a determination the recorded sensor data remaining below the recorded data threshold, set a mode specific medicament requirement to a pre-set constant, wherein the mode specific medicament requirement is used in determining the recommended settings for each parameter in the list of medicament delivery algorithm parameters.

8. The device of claim 7, wherein the processor is further configured to:
determine whether the recorded sensor data is longer than a recorded data threshold; and
in response to a determination the recorded sensor data meeting the recorded data threshold, calculate a mode specific medicament requirement.

9. The device of claim 8, wherein the processor, when calculating the mode specific medicament requirement, is further configured to:
determine a first average that is equal to an average of glucose values during a duration of the recorded data threshold;
determine a second average that is equal to an average of glucose values outside the duration of the recorded data threshold;
determine a percentage difference between the first average and the second average; and
set the mode specific medicament requirement based on the percentage difference.

10. The device of claim 1, wherein the device is further configured to:
select medicament delivery algorithm initial settings.

11. The device of claim 1, wherein the duration of time is a default duration for the initial learning phase of the custom mode.

12. The device of claim 1, wherein during the duration of time, the device is further configured to:
maintain the medicament delivery algorithm parameter settings at a present setting.

13. The device of claim 12, wherein after the duration of time, the device is further configured to:
determine an adjustment value based on the recorded sensor data.

14. The device of claim 13, wherein the adjustment value causes values of the medicament delivery algorithm parameter settings to be adjusted by a percentage derived from the recorded sensor data.

15. A system for establishing a custom mode of a medicament delivery system, comprising:
at least one sensor configured to generate information related to a user;
a wearable medicament delivery device configured to deliver medicament to the user; and
a processor configured to:
receive an indication to establish the custom mode;
evaluate information provided by the at least one sensor to establish medicament delivery algorithm parameter settings of the custom mode; and
generate medicament delivery algorithm parameter settings for the custom mode, wherein the medicament delivery algorithm parameter settings are used by a medicament delivery algorithm that controls the delivery of the medicament to the user.

16. The system of claim 15, wherein the processor is further configured to:
determine whether the recorded sensor data is longer than a recorded data threshold;
in response to a determination the recorded sensor data remaining below the recorded data threshold, set the mode specific medicament requirement to a pre-set constant.

17. The system of claim 15, wherein the processor is further configured to:
determine whether the recorded sensor data is longer than a recorded data threshold;
in response to a determination the recorded sensor data exceeds the recorded data threshold, calculate a mode specific medicament requirement.

18. The system of claim 15, wherein the processor is further configured to:
automatically engage the custom mode at a preset time, preset day, or preset location.

19. The system of claim 15, wherein the at least one sensor includes an analyte sensor, a heart rate monitor, a blood oxygen monitor, a pedometer, a thermometer, chronometer, or location.

20. The system of claim 15, further comprises:
a display device operable to present a graphical user interface, wherein the display device is coupled to the processor, and the graphical user interface is operable to present a name associated with a custom mode to be established.
